(19) United States
(12) Patent Application Publication
(10) Pub. No.: US 2026/0325238 A1
(43) Pub. Date: Oct. 22, 2026

(54) ROBOTIC SYSTEMS AND METHODS FOR ORGAN, TISSUE, AND CELLULAR MONITORING

(71) Applicants: The Trustees of The Stevens Institute of Technology, Hoboken, NJ (US); The Trustees of Columbia University in the City of New York, New York, NY (US)

(72) Inventors: Jinho KIM, Jersey City, NJ (US); Gordana VUNJAK-NOVAKOVIC, New York, NY (US)

(21) Appl. No.: 19/633,298

(22) Filed: Mar. 30, 2026

Related U.S. Application Data
(60) Provisional application No. 63/791,119, filed on Apr. 18, 2025.

(51) Int. Cl. A61B 34/32; G06T 7/00

(52) U.S. Cl. CPC A61B 34/32; G06T 7/0016; G06T 2207/10064; G06T 2207/20081; G06T 2207/30024

(57) ABSTRACT

The present disclosure relates to a robotic, machine learning system for monitoring live biological material. The system includes a visual detection subsystem, processors, and memory with executable instructions. The system receives and analyzes images using a machine learning model to identify target sites and quantify characteristics like cellular density and tissue morphology. The system generates real-time visual representations overlaid on images, continuously monitors changes, provides feedback to user devices, and stores data for future analysis. Methods for monitoring a patient's anatomy during surgery, assisting in re-delivery of surgical instruments to target sites, and automatically guiding surgical instruments based on data are disclosed.

### CROSS-REFERENCE TO RELATED APPLICATIONS

This application claims priority to U.S. Provisional Application No. 63/791,119, filed Apr. 18, 2025, which is hereby incorporated by reference in its entirety.

### STATEMENT REGARDING FEDERALLY SPONSORED RESEARCH

The invention was made with government support under Contract Numbers EB 027062 awarded by the National Institutes of Health (NIH) and 2143620 awarded by the National Science Foundation (NSF). The government has certain rights in the invention.

### TECHNICAL FIELD

The disclosed technology relates to systems and methods for robotic, machine learning organ, tissue, and cellular monitoring. Specifically, the disclosed technology relates to robotic systems that can monitor and learn from prior procedures in order to provide feedback on health or viability of organs, tissues, or cells.

### BACKGROUND

Ex Vivo Lung Perfusion (EVLP) is a technique used to assess and improve donor lungs before transplantation. It involves maintaining the lungs outside the body in a controlled environment. While EVLP has increased the availability and quality of transplantable lungs, it has limitations, primarily due to the lack of continuous, real-time monitoring. Current methods are intermittent and may not detect issues such as edema or inflammation promptly, potentially reducing lung viability. Constant, detailed monitoring during EVLP could provide real-time data, enabling early detection and intervention to improve lung quality. Technologies such as advanced imaging and biochemical sensors could offer comprehensive assessments.

Improved monitoring techniques could also benefit other types of cellular monitoring, or even benefit in vivo applications, such as managing chronic lung conditions, monitoring patients in critical care, or providing support during lung surgery or bronchoscopy exploration. Continuous in vivo monitoring would allow for early complication detection, improved patient outcomes, and personalized treatment plans.

Accordingly, there is a need for improved systems and methods for continuous monitoring that improve lung transplantation outcomes and have potential applications for in vivo lung health management. Embodiments of the present disclosure are directed to these and other considerations.

### SUMMARY

Disclosed embodiments can include a system for robotic, machine learning organ, tissue, and cellular monitoring. In one embodiment, the present disclosure relates to a robotic, machine learning system designed for monitoring live biological materials. This system comprises a visual detection subsystem, one or more processors, and memory that stores instructions executable by the processors. The system is configured to receive images of the live biological material via the visual detection subsystem and analyze these images using a machine learning model to identify target sites within the material. It quantifies characteristics of these target sites, such as cellular density, tissue morphology, or pathological markers, and generates real-time visual representations of these quantified characteristics overlaid on the images. The system continuously monitors changes at the target sites by periodically receiving and reanalyzing updated images, providing feedback to a user device regarding the status or progression of the live biological material, and storing data and feedback for future reference and analysis.

In another embodiment, the present disclosure relates to a method for using data from live biological materials to automatically guide a surgical instrument in locating and positioning itself at a target site. This method involves receiving images associated with the live biological material, analyzing these images to identify the target site, quantifying characteristics of the target site, and continuously monitoring changes at the target site. The system outputs control signals to a motorized system to automatically instruct the surgical instrument to locate and position itself at the target site and adjusts the position of the surgical instrument in real-time based on continuous monitoring and updated analysis.

In another embodiment, the present disclosure relates to a method for monitoring a patient's anatomy during surgery and assisting in the re-delivery of a surgical instrument to a target site. This method includes monitoring the patient's anatomy using a visual detection subsystem, receiving an indication that a surgical instrument has been removed from the target site, analyzing the monitored anatomy to determine the location of the target site, and providing operational guidance to assist in re-delivering the surgical instrument to the same target site. The system adjusts the operational guidance in real-time based on continuous monitoring and updated analysis of the patient's anatomy.

### BRIEF DESCRIPTION OF THE DRAWINGS

Reference will now be made to the accompanying drawings, which are not necessarily drawn to scale, and which illustrate various implementations, aspects, and principles of the disclosed technology. In the drawings:

FIG. 1A is a schematic for an example robotic, machine learning system for monitoring or analyzing target biological material, in accordance with certain embodiments of the disclosed technology.

FIG. 1B shows an experimental setup to train a system on a target, in accordance with certain embodiments of the disclosed technology.

FIG. 2A shows an experimental setup to train a system on a target using light intensity, in accordance with certain embodiments of the disclosed technology.

FIG. 2B shows an experimental setup to train a system on a target using characteristics of an image, in accordance with certain embodiments of the disclosed technology.

FIG. 3A shows an experimental setup wherein fluorescently labeled cells are injected into ex vivo lungs as therapeutics have been imaged, in accordance with certain embodiments of the disclosed technology.

FIG. 3B shows the experimental setup of FIG. 3A wherein the fluorescently labeled cells have been tracked and labeled with a visual representation of the quantified characteristics overlaid on the image of the labeled cells, in accordance with certain embodiments of the disclosed technology.

FIG. 3C is a graph showing the number of the fluorescently labeled cells detected using machine learning-assisted real-time image processing. The X-axis represents time, and the Y-axis represents the number of cells counted.

FIG. 4A is a schematic for an example system that uses data for a biological material to automatically guide a surgical instrument to locate and position itself at a target site, in accordance with certain embodiments of the disclosed technology.

FIG. 4B shows an experimental setup to train a system on a target and move a surgical instrument accordingly, in accordance with certain embodiments of the disclosed technology.

FIG. 4C shows an experimental setup to train a system on a target and move a surgical instrument accordingly, in accordance with certain embodiments of the disclosed technology.

FIG. 4D shows the experimental setup of FIGS. 4B and 4C, wherein the training target is labeled with a visual representation of the quantified characteristics overlaid on the image of the training target, in accordance with certain embodiments of the disclosed technology.

FIG. 5A shows the progression of using target detection and localization using AI, in accordance with certain embodiments of the disclosed technology.

FIG. 5B is a schematic showing the input and output nodes of the target detection and localization using AI, in accordance with certain embodiments of the disclosed technology.

FIG. 6A shows an experimental setup using a machine learning bronchoscope, in accordance with certain embodiments of the disclosed technology.

FIG. 6B shows a setup using the machine learning bronchoscope of FIG. 6A, in accordance with certain embodiments of the disclosed technology.

FIG. 6C shows the steps of loading the AI-driven bronchoscope of FIG. 6B, in accordance with certain embodiments of the disclosed technology.

FIG. 7 shows a machine learning navigation path determination process, in accordance with certain embodiments of the disclosed technology.

FIG. 8A shows a machine learning navigation path determination process, using a first pattern extraction of lung passages, in accordance with certain embodiments of the disclosed technology.

FIG. 8B shows a machine learning navigation path determination process, using a second pattern extraction of lung passages, in accordance with certain embodiments of the disclosed technology.

FIG. 8C shows a machine learning navigation path determination process, using a third pattern extraction of lung passages, in accordance with certain embodiments of the disclosed technology.

FIG. 9A shows a potential navigation path.

FIG. 9B shows the navigation paths of FIG. 9A labeled with a visual representation of the quantified characteristics overlaid on the image of the navigation paths, in accordance with certain embodiments of the disclosed technology.

FIG. 10 is a flow diagram illustrating an exemplary process using a machine learning system for monitoring a biological material, in accordance with certain embodiments of the disclosed technology.

FIG. 11 is a flow diagram illustrating an exemplary method for using data for a live biological material to automatically guide a surgical instrument to locate and position itself at a target site, in accordance with certain embodiments of the disclosed technology.

FIG. 12 is a flow diagram illustrating an exemplary method for monitoring a subject patient's anatomy during a surgery and assisting in re-delivery of a surgical instrument to a target site, in accordance with certain embodiments of the disclosed technology.

FIG. 13 is a block diagram of an example monitoring system for machine learning (“ML”) and/or artificial intelligence (“AI”)-driven organ, tissue, and cellular monitoring, according to an example implementation of the disclosed technology.

FIG. 14 is a block diagram of an example system that can be used to provide one or more systems or methods for monitoring biological material, according to an example implementation of the disclosed technology.

### DETAILED DESCRIPTION

Examples of the present disclosure relate to systems and methods for machine learning (“ML”) and/or artificial intelligence (“AI”)-driven organ, tissue, and cellular monitoring. More particularly, the disclosed technology relates to an AI target tracking system designed for detecting and tracking specific locations in ex vivo organs, such as lungs, or other biological materials based on optical signals generated by the target biological material. The systems and methods described herein utilize, in some instances, machine learning models, which are necessarily rooted in computer technology. These models involve training to complete tasks and make decisions.

The present disclosure details feedback systems that learn markers for damaged or sub-optimal biological materials to monitor and provide valuable feedback on these materials. The system can employ light-intensity-based tracking and pattern recognition to optimize accuracy and speed. It can detect fluorescently labeled cells injected into ex vivo lungs, enabling real-time tracking and quantification of cell numbers, which correlates with dosage. Integrated AI-based target recognition and motor control processes can enhance a robotic endoscopic device, providing flexible manipulation and control in clinical settings. In some embodiments, the robotic endoscopic device may include a cable-driven actuation mechanism with pulleys that enable flexible manipulation and multi-directional movement of the device. Further implementation of AI-based pattern recognition processes can improve device operation accuracy. For example, an AI-coupled motor control technique can use geometric features of specific airways to guide the device toward the target lung region. These systems offer precise, real-time monitoring and control, improving the effectiveness and safety of therapeutic interventions.

In some examples, these systems can involve using visual input data and a machine learning model to teach the characteristics of the target organ, tissue, or cell, and output information about the target or even instructions to move one or more robotic outputs. Using a machine learning model in this way can allow the system to be integrated into existing robotic surgery systems, enhancing the accuracy and speed of diagnosis and treatment during surgery. It can also be incorporated into organ bioreactor systems to enable accurate, continuous, and rapid monitoring of treatment outcomes for ex vivo organs such as lungs, hearts, and livers.

This represents a clear advantage and improvement over prior technologies that required continuous and meticulous monitoring of various parameters such as temperature, pressure, oxygenation, and perfusion flow rates, which demanded highly skilled personnel and sophisticated equipment. Overall, the systems and methods disclosed have significant practical applications in the biological monitoring and preservation field due to their noteworthy improvements in addressing current technological challenges.

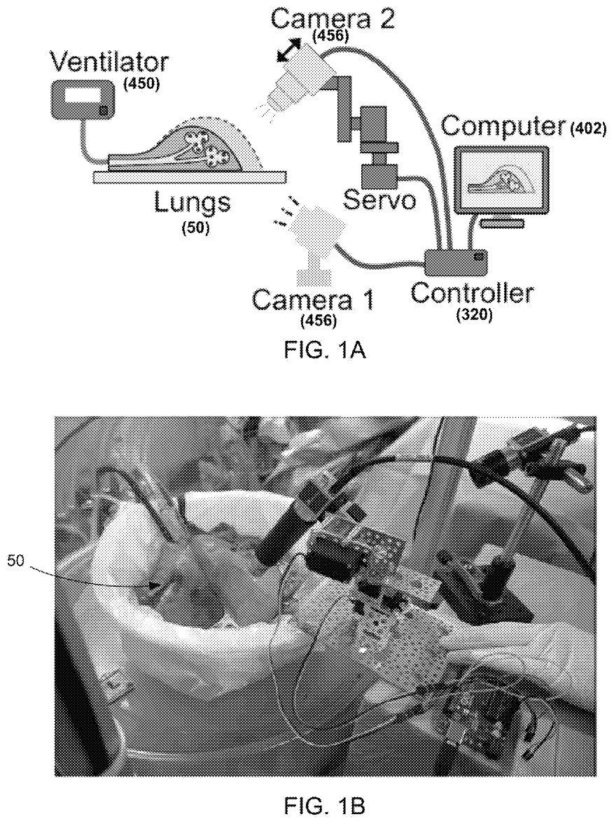

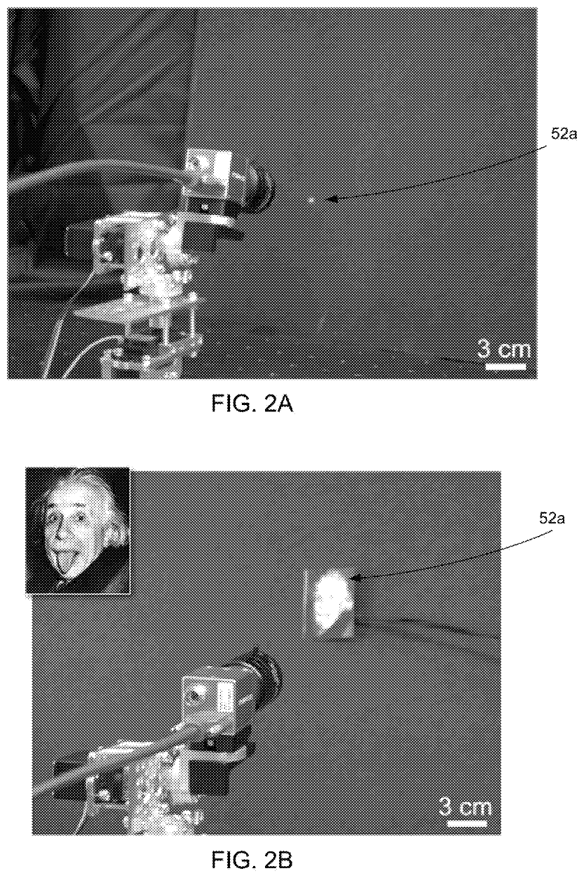

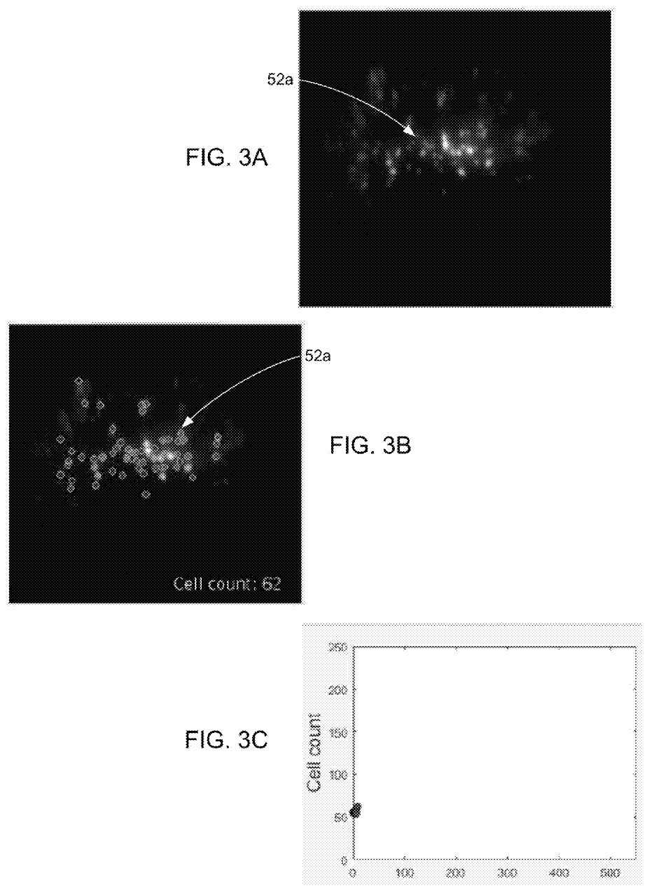

Generally speaking, the improvements to systems for robotic, machine learning organ, tissue, and cellular monitoring can be described at least in terms of two fields of improvement: (i) improvements to imaging and feedback, and (ii) improvements to tracking and delivery. In terms of imaging, the disclosed technology offers several significant benefits. Firstly, the system can utilize optical signals at the target tissue to provide real-time feedback for medical professionals, enabling them to monitor critical parameters such as blood presence and lesions with high precision. An example implementation is shown in the schematic of FIG. 1A, and the in-use example shown in FIG. 1B. This imaging capability is enhanced by the system's ability to analyze color and saturation, allowing for the identification of negative indicators like excessive blood in the airspace. Secondly, the incorporation of light intensity measurements can further refine the imaging process, supporting local fluorescent tracking based on image analysis. An example implementation is shown in the testing setups in FIGS. 2A and 2B. This method enhances the accuracy and detail of the imaging, facilitating better localization and assessment of biological materials. Lastly, the system's fluorescent tracking capabilities are particularly advantageous in surgical applications, such as cancer surgery, where marking specific cells with fluorescent labels allows for precise targeting and improved surgical outcomes. An example implementation is shown in the FIGS. 3A and 3B, with FIG. 3C showing the cell numbers counted using machine learning-assisted real-time image processing, where the X-axis represents time and the Y-axis represents the number of cells counted. Additionally, this tracking can monitor therapeutic agent dosages at specific sites, ensuring accurate treatment delivery. These imaging improvements collectively enhance the system's ability to provide detailed, accurate, and real-time monitoring and feedback, significantly improving the effectiveness and safety of medical interventions. Another benefit of the present systems and methods is that they can account for tissues that are moving during monitoring or some other procedure. For example, lungs continuously move during ventilation, making it difficult to track portions of the lung that are of interest, such as blood, edema, or other damages, especially when tracking with a microscope. Similarly, a heart or other organ can move when a perfusion pump is employed, making it also difficult to track portions of the organ. The present systems and methods can account for such movement and can also be used to output to other devices, such as robotic instruments or endoscopic devices, so that the instrument and/or device can account for the movement.

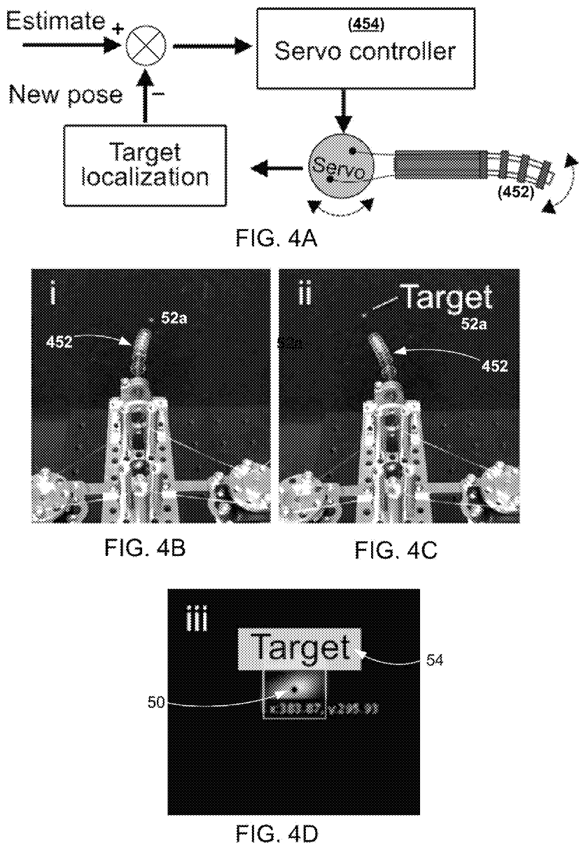

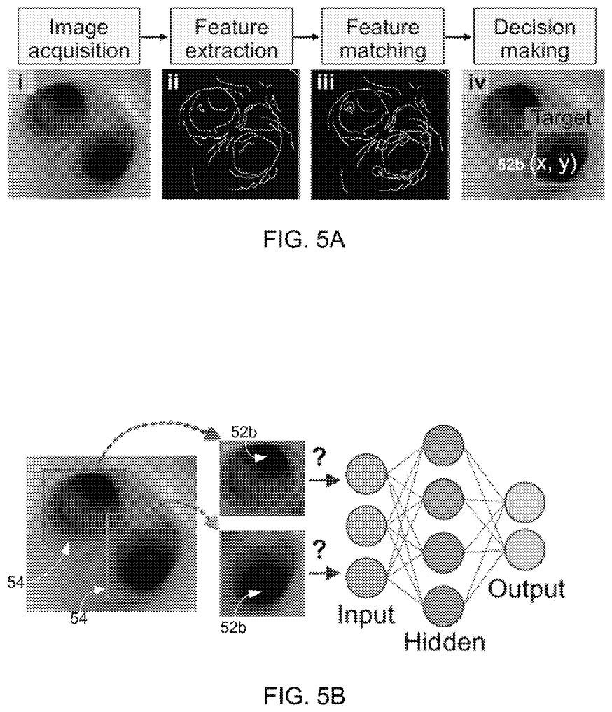

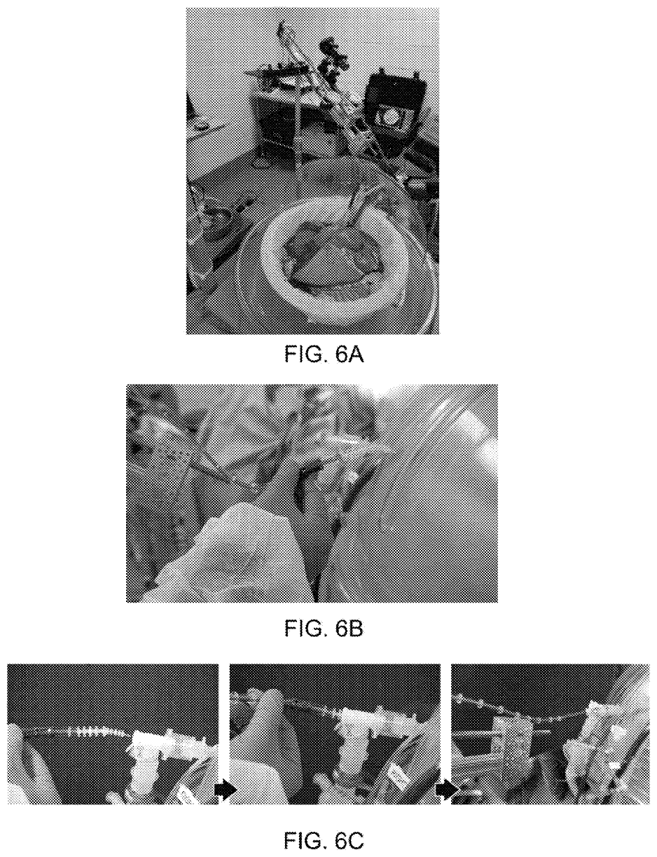

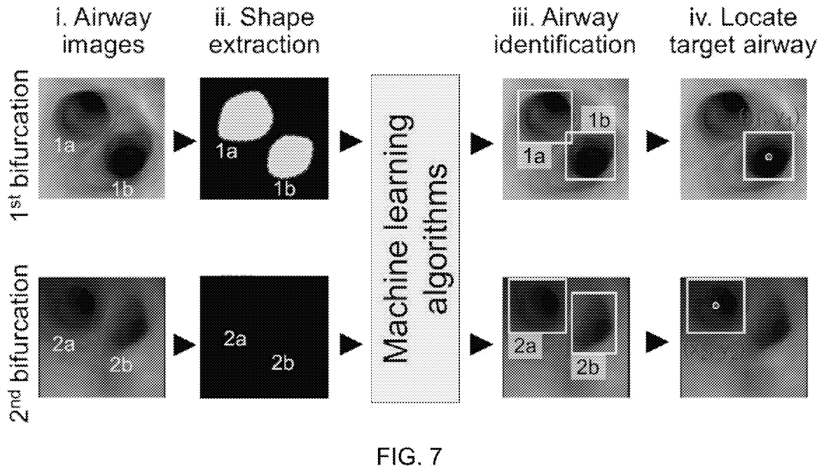

In terms of tracking and delivery, the disclosed technology provides several notable advancements. The system's motor driving capabilities enable in vivo monitoring during surgery, allowing for precise detection and tracking of desired locations. This approach enables the accurate three-dimensional localization of target objects, such as cells, tissues, or organs. An example implementation is shown in the schematic of FIG. 4A, and the in-use example is shown in FIGS. 4B-4C, and FIG. 4D shows the identification of the target via the detection of the light contrast in the view of the system. This is particularly beneficial in procedures where a catheter must be removed and then accurately replaced, as the system can utilize personalized and individual anatomical mapping to guide the catheter back to the correct location. Additionally, the system's pattern detection capabilities, which can be trained on MRI or x-ray images, enhance its ability to recognize and respond to specific anatomical features. An example implementation is shown in FIGS. 5A and 5B. Machine testing further supports this by ensuring the reliability and accuracy of the pattern detection processes. FIGS. 6A-6C show examples of a machine testing setup for an AI-coupled robotic bronchoscope. The automatic delivery feature, as shown in FIGS. 7-9B, addresses challenges faced by pulmonologists in navigating to specific lung areas. By training the system to remember a patient's unique anatomy, it can facilitate easier and more accurate access to targeted regions, even if the device needs to be removed and reinserted. This capability can significantly improve the efficiency and effectiveness of therapeutic interventions, ensuring that treatments are delivered precisely where needed.

Some implementations of the disclosed technology will be described more fully with reference to the accompanying drawings. This disclosed technology can, however, be embodied in many different forms and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods.

Reference will now be made in detail to example embodiments of the disclosed technology that are illustrated in the accompanying drawings and disclosed herein. Wherever convenient, the same reference numbers will be used throughout the drawings to refer to the same or like parts.

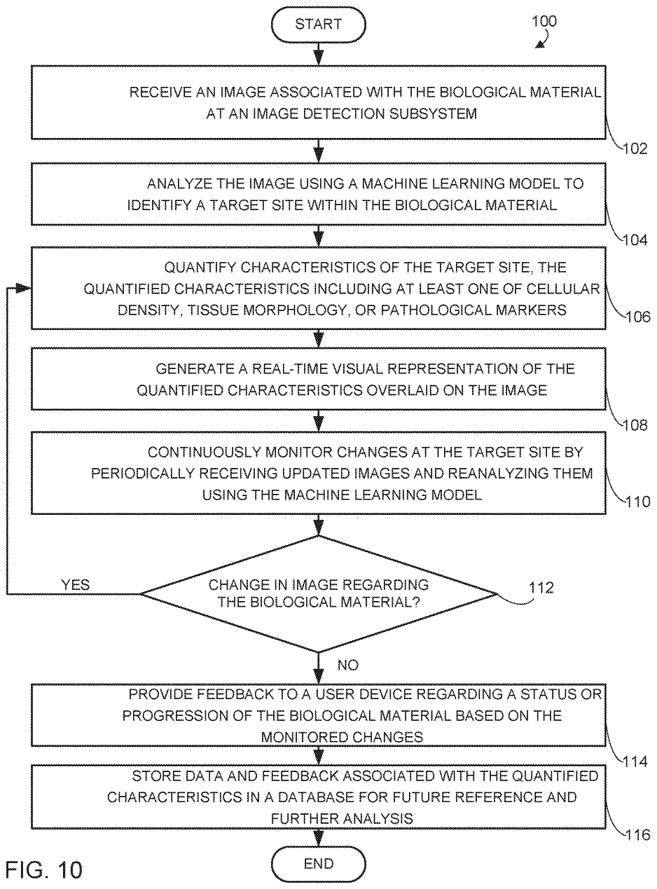

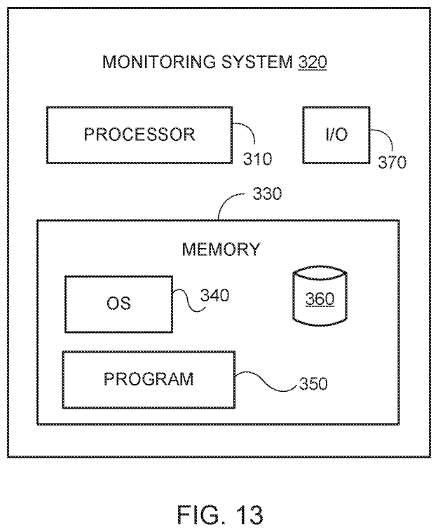

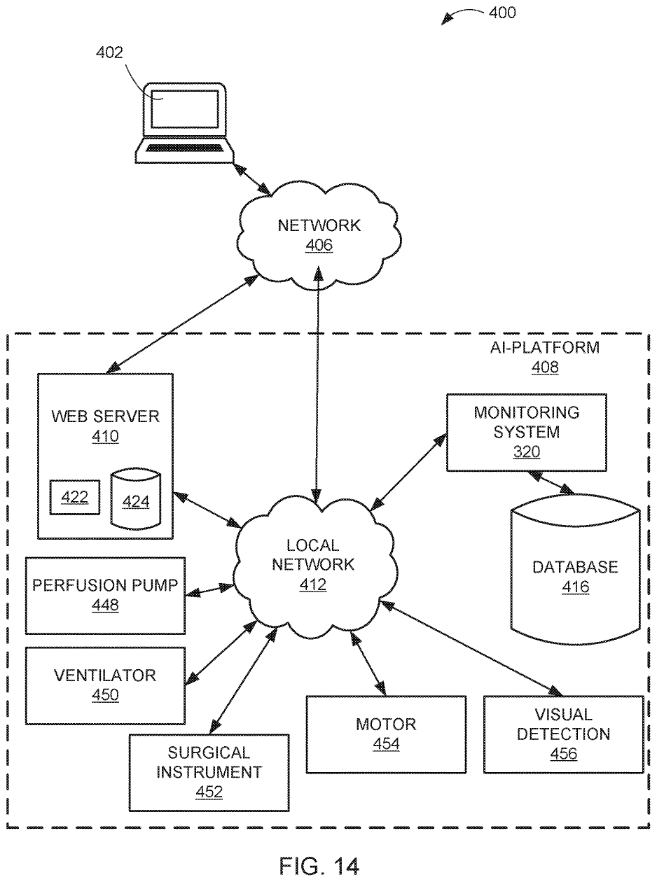

FIG. 10 is a flow diagram illustrating an exemplary process 100 for monitoring a live biological material, such as biological material 50 in FIGS. 1A and 1B, which can include a cell, tissue and/or organ, in accordance with certain embodiments of the disclosed technology. The steps of process 100 can be performed by one or more components of the system 400 (e.g., Monitoring System 320 or web server 410 of AI-Platform 408 or user device 402), as described in more detail with respect to FIGS. 13 and 14.

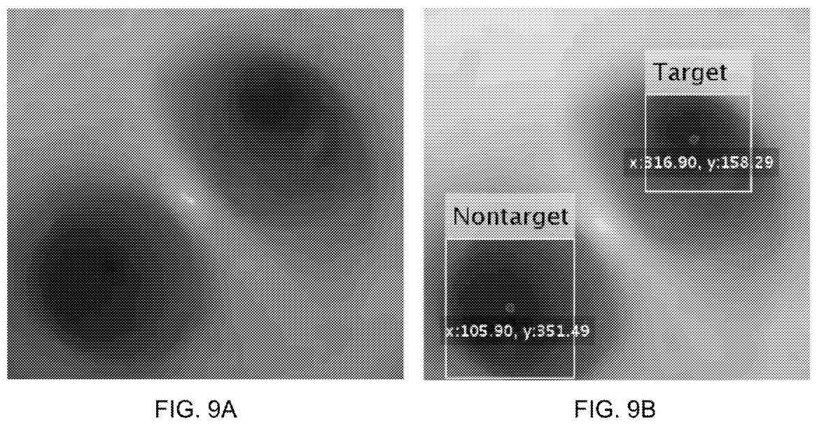

At block 102, process 100 can include receiving, at the one or more processors 310 and via the visual detection subsystem (e.g., visual detection subsystem 456 in FIG. 14), an image associated with the live biological material 50. The image may be captured by the visual detection subsystem 456 and transmitted to the one or more processors 310 for further analysis. Referring to FIGS. 1A and 1B for example, the system in FIG. 1A shows an example of one or more cameras (e.g., visual detection subsystem 456) that can be used to monitor the biological material 50 in FIG. 2B. Similarly, FIGS. 5A and 5B show detection of live biological material, but in the context of using biological material (e.g., airway passages) to train the system by (i) acquiring an image, (ii) extracting features from the image, (iii) matching features that are similar, and (iv) identifying targets using the image gathered (e.g., using nodes of a neural network as shown in FIG. 5B). A similar method of training a system based on biological material (again airway passages) is shown in FIG. 7, and again in FIG. 9A (showing the passages) and 9B (showing the targets identified). Other use cases of receiving an image associated with the live biological material 50, as in block 102, is identifying therapeutic agents at a target site. The step of “training” the system can be performed in the following step in block 104.

At block 104, process 100 can include analyzing, via the one or more processors 310, the image using a machine learning model to identify a target site 52a, 52b within the live biological material 50. The machine learning model may be trained to recognize specific features or patterns indicative of the target site 52a, 52b. To clarify, reference to target site 52a will be understood to mean a training target site (see, e.g., FIGS. 2A, 2B, 3A, and 3B); reference to target site 52b will be understood to mean a biological target site (see, e.g., FIGS. 5A and 5B). Reference target sites can be used to train the system based on, for example, light intensity or contrast (e.g., FIGS. 2A and 2B) or pattern recognition schemes (e.g., FIGS. 5A and 5B). In one embodiment, the system 320, 408 can be designed to identify a dosage of a therapeutic agent at a target site 52b. The system accomplishes this by using the light intensity or fluorescence detected in the image of the target site 52a, 52b, as shown in FIGS. 3A-3C. In FIG. 3A, fluorescently labeled cells are injected into ex vivo lungs as therapeutics, and in FIG. 3B the cells are tracked to train the system. FIG. 3C shows the fluorescently labeled cells being detected and counted in real time using machine learning-based image processing. The X-axis of the graph represents time, and the Y-axis of the graph represents the cell number determined. In some embodiments, the system can track specific cell populations, including implanted cells such as stem cells, as well as endogenous cells that have been modified to express optical signals. The system may monitor these cells to track their differentiation, therapeutic activities, or cell death over time. The system can be configured to recognize trends related to injection parameters, which are associated with the different therapeutics and target organs. Additionally, the injection parameters associated with the various therapeutics and target organs include at least one of dosage, target organ, or injection site.

Furthermore, the system can receive intraoperative input regarding at least one of the cellular density, cell viability, tissue morphology, pathological markers, or organ function; and retrain the machine learning model based on the intraoperative input. For example, nurses or other personnel in an operation can monitor the status of the biological material 50 and input information about the status into a user device 402. This information could be information about pooling blood, tissue damage, edema, or the like, as the system 320, 408 can then develop an understanding of when underlying issues are identified in biological material. This dynamic retraining allows the system to adapt in real-time, enhancing its accuracy and effectiveness in identifying and targeting specific sites within the biological material.

At block 106, process 100 can include quantifying, via the one or more processors 310, characteristics of the target site 52a, 52b. The quantified characteristics can include at least one of cellular density, tissue morphology, or pathological markers. This quantification can be a part of the training of the system 320, 408. For example, the system may analyze images to determine the density of fluorescently labeled cells within a specific region of an ex vivo lung. This information can be used to assess the effectiveness of cell-based therapies by correlating cell density with therapeutic outcomes. Additionally, the system can evaluate tissue morphology by examining structural features of the tissue, such as the integrity of alveolar walls in lung tissue or the presence of fibrosis. By quantifying these morphological characteristics, the system can provide insights into the health and viability of the tissue, which can be useful for applications such as organ transplantation or regenerative medicine. Pathological markers, such as the presence of specific proteins or abnormal cell types, can also be quantified. For instance, the system may detect and measure the expression levels of biomarkers associated with cancer or inflammation. This capability allows for the early detection of pathological changes and the monitoring of disease progression or response to treatment. In another example, during a surgical procedure, the system can quantify the extent of tissue damage by analyzing the intensity and distribution of optical signals from injured areas. This real-time quantification can guide surgeons in making precise interventions, such as removing damaged tissue while preserving healthy areas. Furthermore, the system can be trained to recognize and quantify therapeutic agent dosages at the target site. For example, by measuring the fluorescence intensity of labeled therapeutic agents, the system can determine the concentration and distribution of the agents within the tissue. This information is helpful to ensure accurate and effective delivery of treatments, such as targeted drug delivery or gene therapy.

At block 108, process 100 can include generating, via the one or more processors 310, a real-time visual representation 54 of the quantified characteristics overlaid on the image. This visual representation 54 may provide a detailed view of the target site 52a, 52b and its quantified characteristics, see, for example, the examples shown in FIGS. 4D and 5B. At block 110, process 100 can include continuously monitoring, via the one or more processors 310, changes at the target site 52a, 52b by periodically receiving updated images and reanalyzing the updated images using the machine learning model. This continuous monitoring allows for real-time tracking of changes in the target site 52a, 52b, enabling the system to detect and respond to dynamic alterations in the biological material.

For example, in the context of monitoring an ex vivo lung, the system can periodically capture images of the lung tissue and analyze these images to detect changes in cellular density. If the system identifies a decrease in cellular density in a specific region, it may indicate cell death or tissue degradation. The system can then generate a visual representation highlighting this region, allowing medical professionals to take appropriate action, such as adjusting the perfusion parameters or applying targeted therapies.

In another example, during a surgical procedure involving the removal of a tumor, the system can continuously monitor the surgical site to detect any residual cancerous cells. By periodically capturing and analyzing images of the tissue, the system can identify areas with abnormal fluorescence or other pathological markers indicative of cancer. The real-time visual representation can guide the surgeon in ensuring complete removal of the tumor, thereby reducing the risk of recurrence.

Additionally, in the context of organ transplantation, the system can be used to monitor the health of an ex vivo heart. The system can periodically capture images of the heart tissue and analyze these images to detect changes in tissue morphology, such as the development of fibrosis or other structural abnormalities. The continuous monitoring process can provide real-time feedback on the condition of the heart, helping transplant surgeons make informed decisions about the viability of the organ for transplantation. At block 112, process 100 can include determining, via the one or more processors 310, if there is a change in the image regarding the biological material 50. If a change is detected, the process can return to block 106 for re-quantification of characteristics. If no change is detected, the process proceeds to block 114.

At block 114, process 100 can include providing feedback, via the one or more processors 310, to a user device 402 regarding the status or progression of the live biological material 50 based on the monitored changes. This feedback can include visual, textual, or auditory information to inform the user of the current state of the biological material 50. For example, in a surgical setting, the system can display a real-time visual overlay on a monitor, highlighting areas of concern such as regions with abnormal fluorescence or changes in tissue morphology. Textual feedback might include alerts or notifications about significant changes in cellular density or the presence of pathological markers. Auditory feedback could involve alarms or spoken updates to ensure that the surgical team is immediately aware of critical changes.

At block 116, process 100 can include storing data and feedback associated with the quantified characteristics in a database 360, 416 for future reference and further analysis. This stored data can include images, quantified metrics, and historical changes observed during the monitoring process. For instance, if the system is used to monitor the progression of a disease in an ex vivo organ, the stored data can provide a comprehensive record of how the organ's condition has evolved over time. Additionally, the stored data can be used to refine and improve the machine learning model. By analyzing historical data, the system can identify patterns and trends that may not be immediately apparent, leading to more accurate and reliable predictions in future applications. For example, if the system consistently detects certain changes in tissue morphology that precede pathological developments, this information can be used to enhance the model's ability to predict and respond to similar changes in other cases.

The process 100 can end after block 116. However, in other embodiments, additional steps can be performed in accordance with the present disclosure. For example, the process 100 performed by the system 320, 408 can include receiving information regarding (i) ventilator or perfusion pump usage data, and (ii) monitoring information related to movement of the live biological material, and updating the system to account for the movement of the live biological material 50 to predict a location of the live biological material 50 at a particular time based on the ventilator (see ventilator 450 in FIG. 14) or perfusion pump (see perfusion pump 448 in FIG. 14) usage data and the monitoring information. For example, during an ex vivo lung perfusion procedure, the system can receive data from the ventilator regarding the frequency and intensity of breaths being simulated. Concurrently, it can monitor the movement of the lung tissue using imaging techniques. By integrating this data, the system can predict the lung's position and movement patterns, allowing for more precise monitoring and intervention. This capability is particularly beneficial in scenarios where real-time adjustments are critical, such as during surgical procedures or in the management of dynamic biological systems.

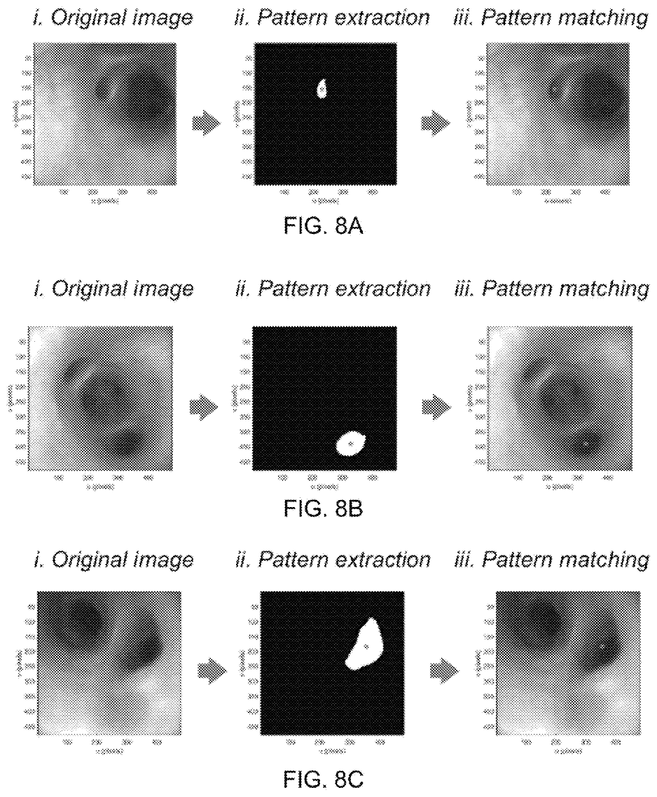

In some examples, process 100 can include training the machine learning model on medical imaging, wherein the medical imaging can include at least one of computed tomography scans (CT scans), magnetic resonance imaging (MRIs), X-rays, ultrasounds, or nuclear medicine. For instance, CT scans and MRIs provide detailed cross-sectional images of the body, which can be used to train the system to recognize and differentiate between various tissues and organs. X-rays offer a quick and effective way to visualize bone structures and detect fractures or abnormalities. Ultrasounds are particularly useful for real-time imaging of soft tissues and organs, such as the heart and liver, while nuclear medicine techniques can highlight metabolic processes and identify areas of abnormal activity. The system can identify passages through patient anatomy, the passages including at least one of vessels, bronchi, bronchioles, tracheas, or alveoli. Such examples are highlighted in FIGS. 7-8C. FIG. 7 shows two separate bifurcations that are processed to identify airways. FIGS. 8A, 8B, and 8C are each examples of pattern extraction to identify airways. In the context of pulmonary medicine, the system can analyze imaging data to map out the bronchial tree, identifying the main bronchi, smaller bronchioles, and alveolar sacs. This detailed anatomical mapping can assist pulmonologists in navigating to specific lung regions during bronchoscopy, ensuring accurate biopsy or treatment delivery. Similarly, in cardiovascular applications, the system can identify and map blood vessels, aiding in procedures such as angioplasty or stent placement.

By training on various medical imaging modalities, the system can create personalized anatomical maps for each patient. These maps can be used to plan and execute personalized treatment plans. For instance, in cancer treatment, the system can identify the exact location and extent of a tumor using MRI and CT scans, and then guide the delivery of radiation therapy to target the tumor precisely while sparing healthy tissue. The integration of multiple imaging modalities can enhance the system's diagnostic capabilities. For example, combining data from CT scans and PET scans can provide both anatomical and functional information, allowing for a comprehensive assessment of conditions such as cancer or cardiovascular disease. This multimodal approach can improve the accuracy of diagnoses and the effectiveness of subsequent treatments. The system's ability to continuously monitor and provide real-time feedback is particularly beneficial in critical care settings. For example, in an intensive care unit (ICU), the system can continuously monitor a patient's lung function using data from ventilators and imaging systems. Any changes in the patient's condition can be detected immediately, allowing for prompt intervention and adjustment of treatment plans. By incorporating these advanced capabilities, the disclosed system represents a significant advancement in the field of medical monitoring and intervention, offering improved accuracy, real-time adaptability, and personalized care.

In some embodiments, the system 320, 408 can create a registry of information to predict anatomical features of a patient. This registry can be used to enhance the accuracy of the system's predictions and provide a comprehensive database of anatomical information. The system 320, 408 can also provide operational guidance based on the registry during an operation. This guidance can be output for display on a user device 402, allowing medical professionals to access real-time information and make informed decisions during surgical procedures. For example, the system can display a detailed map of the patient's anatomy, highlighting critical structures and providing step-by-step guidance for navigating complex anatomical regions. This feature is particularly useful in minimally invasive surgeries, where precise navigation is crucial for successful outcomes. In some embodiments, the controller may be implemented using a microcontroller, such as an Arduino board, for servo motor control.

In some embodiments, the system 320, 408 can store instructions configured to train the machine learning model to analyze and store a patient's anatomical data. The system can receive an indication that a surgical instrument 452 (see FIG. 14) has been removed from the target site 52b in the patient and provide operational guidance to a user when the user re-inserts the surgical instrument 452, assisting in placing the surgical instrument 452 back at the target site 52b. The system 320, 408 can store instructions configured to output a control signal to a motor to automatically locate and position a surgical instrument 452 at the target site 52b within the live biological material 50.

For example, a device embodying such features was tested during bronchoscopic procedures using ex vivo swine lungs. The navigation path was determined by the AI-coupled motor control technique, which used the geometric features of specific airways to guide the device toward the target lung region. FIGS. 6A to 6C show such an example. When the device is retracted (e.g., FIG. 6B), the system has a previously identified mapping of the patient's specific anatomy. When a scope is inserted back into the patient, it is already trained to know where the device is to move to target the target site 52b.

This demonstrates the system's ability to accurately navigate the bronchial pathways of the swine lungs, even after the scope was removed and reinserted. The AI model, trained on the geometric features of the airways, allows the system to predict and adjust for the movement of the lungs, ensuring that the scope could be guided back to the precise location of the target site 52b. This feature is particularly useful in clinical settings where maintaining the exact positioning of surgical instruments is critical for the success of the procedure and the safety of the patient.

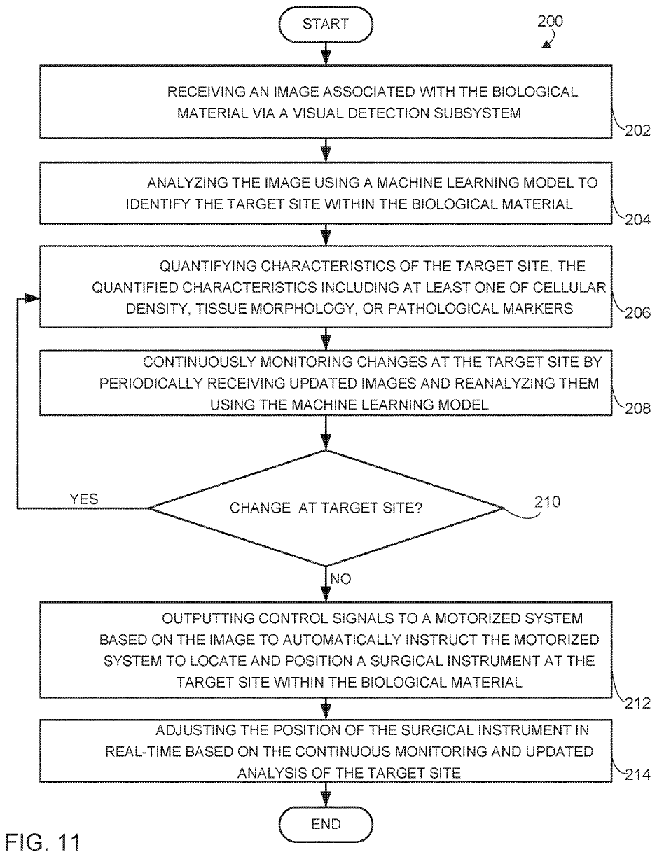

FIG. 11 is a flow diagram illustrating an exemplary method 200 for using data for a live biological material 50 to automatically assist a surgical instrument 452 to locate and position itself at a target site 52a, 52b, in accordance with certain embodiments of the disclosed technology. The steps of method 200 can be performed by one or more components of the system 400 (e.g., Monitoring System 320 or web server 410 of AI-Platform 408 or user device 402), as described in more detail with respect to FIGS. 13 and 14. Method 200 of FIG. 11 is similar to process 100 of FIG. 10. The descriptions of blocks 202, 204, 206, and 208, in method 200 are similar to the respective descriptions of blocks 102, 104, 106, and 110 of process 100, respectively, and are not repeated below. However, block 214 is different from block 114 and is described below. Additional blocks 212 and 214 are also described below. In block 210, the system 320, 408 determines if there is a change in the image regarding the target site 52a, 52b. If a change is detected, the process can return to block 206 for re-quantification of characteristics. If no change is detected, the process proceeds to block 212.

At block 212, method 200 can include outputting, via one or more processors (e.g., processor 310), control signals to a motorized system (e.g., motor 454 in FIG. 14) based on the image detected to automatically instruct the motorized system to locate and position a surgical instrument 452 at a target site 52a, 52b within the live biological material 50. This step leverages the machine learning model's ability to analyze real-time imaging data and generate precise control signals that guide the motorized system. The control signals are derived from the continuous monitoring and analysis of the live biological material, ensuring that the surgical instrument is accurately positioned at the target site. At block 214, method 200 can include adjusting, via the one or more processors, the position of the surgical instrument 452 in real-time based on the continuous monitoring and updated analysis of the target site 52a, 52b. This step leverages the machine learning model's ability to analyze real-time imaging data and generate precise control signals that guide the motorized system. The control signals are derived from the continuous monitoring and analysis of the live biological material, ensuring that the surgical instrument is accurately positioned at the target site. For example, during bronchoscopic procedures using ex vivo swine lungs, the AI-coupled motor control technique utilized geometric features of specific airways to guide the device toward the target lung region. FIGS. 6A to 6C illustrate this process. When the device is retracted (e.g., FIG. 6B), the system relies on a previously identified mapping of the patient's specific anatomy. When the scope is reinserted into the patient, the system is already trained to navigate to the target site 52b. This capability ensures that the surgical instrument can be accurately repositioned, even after removal, by utilizing the stored anatomical data and real-time imaging feedback. The system's ability to output control signals to a motorized system enhances the precision and efficiency of surgical procedures. By automating the positioning of surgical instruments, the system reduces the risk of human error and improves the overall outcome of the surgery. This feature is particularly beneficial in complex procedures where maintaining the exact positioning of instruments is critical for patient safety and procedural success.

The method 200 can end after block 214. However, in other embodiments, additional steps can be performed in accordance with the present disclosure. For example, method 200 can include receiving, at the one or more processors, information regarding (i) ventilator or perfusion pump usage data, and (ii) monitoring information related to movement of the live biological material 50. The system can then update, via the one or more processors, to account for the movement of the live biological material 50 to predict a location of the live biological material 50 at a particular time based on the ventilator or perfusion pump usage data and the monitoring information. This capability is particularly beneficial in scenarios where real-time adjustments are critical, such as during surgical procedures or in the management of dynamic biological systems. For example, during an ex vivo lung perfusion procedure, the system can receive data from the ventilator regarding the frequency and intensity of breaths being simulated. Concurrently, it can monitor the movement of the lung tissue using imaging techniques. By integrating this data, the system can predict the lung's position and movement patterns.

Method 200 can include training the machine learning model to analyze and store a person's anatomical data. The system can receive, via the one or more processors, an indication that a surgical instrument 452 has been removed from the target site 52a, 52b in the person. The system can then provide operational guidance, via the one or more processors 310, to a user when the user re-inserts the surgical instrument 452 to assist in placing the surgical instrument 452 back at the target site 52a, 52b. This feature ensures that the surgical instrument can be accurately repositioned, even after removal, by utilizing the stored anatomical data and real-time imaging feedback.

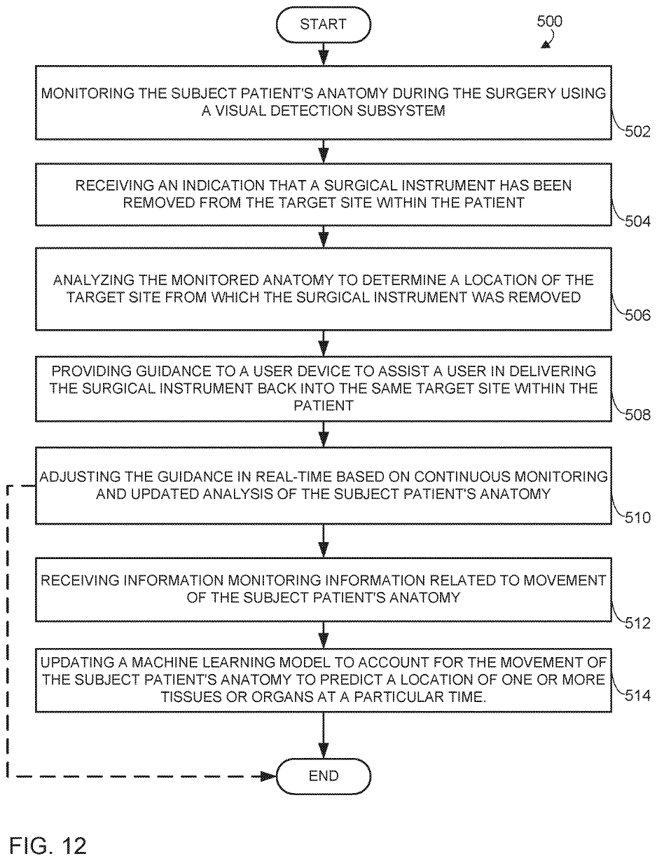

FIG. 12 is a flow diagram illustrating an exemplary Method 500 for monitoring a subject patient's anatomy during a surgery and assisting in re-delivery of a surgical instrument 452 to a target site, in accordance with certain embodiments of the disclosed technology. The steps of method 500 can be performed by one or more components of the system 400 (e.g., Monitoring System 320 or web server 410 of AI-Platform 408 or user device 402), as described in more detail with respect to FIGS. 13 and 14. At block 502, method 500 can include monitoring, via one or more processors (e.g., processor 310), the subject patient's anatomy during the surgery using a visual detection subsystem 456. This subsystem can include cameras or other imaging devices that capture real-time images of the patient's internal structures. The captured images are transmitted to the one or more processors 310 for analysis. In this step, the visual detection subsystem 456 can be equipped with various advanced imaging technologies, including high-resolution cameras, endoscopic devices, optical coherence tomography (OCT), and fluorescence imaging. Additionally, it can incorporate ultrasound imaging, magnetic resonance imaging (MRI), computed tomography (CT), X-ray imaging, nuclear medicine imaging, infrared imaging, confocal microscopy, and photoacoustic imaging. These devices can provide detailed and dynamic images of the surgical field, allowing the system to continuously monitor the patient's anatomy. The real-time images can be processed using advanced image processing algorithms to enhance clarity, contrast, and detail. This can assist in ensuring that the system accurately captures the anatomical features and any changes that occur during the surgery.

At block 504, the method can include receiving, at the one or more processors 310, an indication that a surgical instrument 452 has been removed from the target site 52a, 52b within the patient. This indication can be generated by sensors or input from the surgical team, e.g., surgeon, surgical technician, nurse, and the like. The indication that a surgical instrument has been removed can be detected through various means, such as force sensors, position sensors, or manual input from the surgical team. For example, the surgical instrument 452 can be equipped with sensors that detect changes in pressure or position, signaling its removal from the target site. Alternatively, the surgical team can manually input this information into the system via a user interface. This step ensures that the system is aware of the instrument's status and can take appropriate actions to assist in its re-delivery. At block 506, the method can include analyzing, via the one or more processors 310, the monitored anatomy to determine a location of the target site 52a, 52b from which the surgical instrument 452 was removed. This analysis involves comparing the current anatomical images with previously stored images and data to accurately identify the target site. The analysis of the monitored anatomy involves using machine learning algorithms and image recognition techniques to compare the real-time images with a database of preoperative and intraoperative images. The system can utilize pattern recognition, feature extraction, and image registration techniques to identify the exact location of the target site 52a, 52b. This process can also involve the use of 3D reconstruction and mapping to create a detailed model of the patient's anatomy, allowing for precise localization of the target site.

At block 508, the method can include providing operational guidance, via the one or more processors 310, to a user device 402 to assist a user in delivering the surgical instrument 452 back into the same target site 52a, 52b within the patient. This guidance can be in the form of visual overlays, textual instructions, or auditory cues that direct the user to the precise location of the target site. The operational guidance provided to the user can be highly interactive and intuitive. For example, the system can generate augmented reality (AR) overlays on a display screen, highlighting the path and target site for the surgical instrument. Textual instructions can provide step-by-step guidance, while auditory cues can alert the user to important actions or adjustments. This multimodal guidance ensures that the user receives comprehensive support in re-delivering the instrument to the target site.

At block 510, the method can include adjusting, via the one or more processors 310, the operational guidance in real-time based on continuous monitoring and updated analysis of the subject patient's anatomy. This ensures that any changes in the patient's anatomy, such as movement or deformation of tissues, are accounted for, and the surgical instrument is accurately guided back to the target site 52a, 52b. Real-time adjustments to the operational guidance are crucial for maintaining accuracy and safety during surgery. The system continuously monitors the patient's anatomy and updates the guidance based on any detected changes. This can involve compensating for movements caused by breathing, heartbeat, or other physiological factors. The system can use predictive modeling and adaptive algorithms to anticipate and correct for these movements, ensuring that the surgical instrument remains on the correct path to the target site. After block 510, the method 500 can end, or it can continue to block 512. At block 512, the method can include receiving, at the one or more processors 310, information regarding (i) ventilator or perfusion pump usage data, and (ii) monitoring information related to movement of the live biological material 50. The system can then update, via the one or more processors 310, to account for the movement of the live biological material 50 to predict a location of the live biological material 50 at a particular time based on the ventilator or perfusion pump usage data and the monitoring information. This capability is particularly beneficial in scenarios where real-time adjustments are critical, such as during surgical procedures or in the management of dynamic biological systems. For example, during an ex vivo lung perfusion procedure, the system can receive data from the ventilator regarding the frequency and intensity of breaths being simulated. Concurrently, it can monitor the movement of the lung tissue using imaging techniques. By integrating this data, the system can predict the lung's position and movement patterns, allowing for more precise monitoring and intervention.

At block 514, the method can include training the machine learning model to analyze and store a person's anatomical data, or rather can include updating a machine learning model to account for the movement of the subject patient's anatomy to predict a location of one or more tissues or organs at a particular time. The system can receive, via the one or more processors 310, an indication that a surgical instrument 452 has been removed from the target site 52a, 52b in the person. The system can then provide operational guidance, via the one or more processors 310, to a user when the user re-inserts the surgical instrument 452 to assist in placing the surgical instrument 452 back at the target site 52a, 52b. This feature ensures that the surgical instrument can be accurately repositioned, even after removal, by utilizing the stored anatomical data and real-time imaging feedback. This capability is particularly useful in clinical settings where maintaining the exact positioning of surgical instruments is critical for the success of the procedure and the safety of the patient.

The method 500 can end after block 514. However, in other embodiments, additional steps can be performed in accordance with the present disclosure, including for example creating a registry of information to predict anatomical features of a patient. This registry can be used to enhance the accuracy of the system's predictions and provide a comprehensive database of anatomical information.

FIG. 13 is a block diagram of an example monitoring system 320 for ML and/or AI-driven organ, tissue, and cellular monitoring, according to an example implementation of the disclosed technology. According to some embodiments, the user device 402 and web server 410, as depicted in FIG. 14 and described below, can have a similar structure and components that are similar to those described with respect to Monitoring System 320 shown in FIG. 13. As shown, the Monitoring System 320 can include a processor 310, an input/output (I/O) device 370, a memory 330 containing an operating system (OS) 340 and a program 350. In certain example implementations, the Monitoring System 320 can be a single server or can be configured as a distributed computer system including multiple servers or computers that interoperate to perform one or more of the processes and functionalities associated with the disclosed embodiments. In some embodiments Monitoring System 320 can be one or more servers from a serverless or scaling server system. In some embodiments, the Monitoring System 320 can further include a peripheral interface, a transceiver, a mobile network interface in communication with the processor 310, a bus configured to facilitate communication between the various components of the Monitoring System 320, and a power source configured to power one or more components of the Monitoring System 320.

A peripheral interface, for example, can include the hardware, firmware and/or software that enable(s) communication with various peripheral devices, such as media drives (e.g., magnetic disk, solid state, or optical disk drives), other processing devices, or any other input source used in connection with the disclosed technology. In some embodiments, a peripheral interface can include a serial port, a parallel port, a general-purpose input and output (GPIO) port, a game port, a universal serial bus (USB), a micro-USB port, a high-definition multimedia interface (HDMI) port, a video port, an audio port, a Bluetooth™ port, a near-field communication (NFC) port, another like communication interface, or any combination thereof.

In some embodiments, a transceiver can be configured to communicate with compatible devices and ID tags when they are within a predetermined range. A transceiver can be compatible with one or more of: radio-frequency identification (RFID), near-field communication (NFC), Bluetooth™, low-energy Bluetooth™ (BLE), WiFi™, ZigBee™, ambient backscatter communications (ABC) protocols or similar technologies.

A mobile network interface can provide access to a cellular network, the Internet, or another wide-area or local area network. In some embodiments, a mobile network interface can include hardware, firmware, and/or software that allow(s) the processor(s) 310 to communicate with other devices via wired or wireless networks, whether local or wide area, private or public, as known in the art. A power source can be configured to provide an appropriate alternating current (AC) or direct current (DC) to power components.

The processor 310 can include one or more of a microprocessor, microcontroller, digital signal processor, co-processor or the like or combinations thereof capable of executing stored instructions and operating upon stored data. The memory 330 can include, in some implementations, one or more suitable types of memory (e.g. such as volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like), for storing files including an operating system, application programs (including, for example, a web browser application, a widget or gadget engine, and or other applications, as necessary), executable instructions and data. In one embodiment, the processing techniques described herein can be implemented as a combination of executable instructions and data stored within the memory 330.

The processor 310 can be one or more known processing devices, such as, but not limited to, a microprocessor from the Core™ family manufactured by Intel™, the Ryzen™ family manufactured by AMD™, or a system-on-chip processor using an ARM™ or other similar architecture. The processor 310 can constitute a single core or multiple core processor that executes parallel processes simultaneously, a central processing unit (CPU), an accelerated processing unit (APU), a graphics processing unit (GPU), a microcontroller, a digital signal processor (DSP), a field-programmable gate array (FPGA), an application-specific integrated circuit (ASIC) or another type of processing component. For example, the processor 310 can be a single core processor that is configured with virtual processing technologies. In certain embodiments, the processor 310 can use logical processors to simultaneously execute and control multiple processes. The processor 310 can implement virtual machine (VM) technologies, or other similar known technologies to provide the ability to execute, control, run, manipulate, store, etc. multiple software processes, applications, programs, etc. One of ordinary skill in the art would understand that other types of processor arrangements could be implemented that provide for the capabilities disclosed herein.

In accordance with certain example implementations of the disclosed technology, the Monitoring System 320 can include one or more storage devices configured to store information used by the processor 310 (or other components) to perform certain functions related to the disclosed embodiments. In one example, the Monitoring System 320 can include the memory 330 that can include instructions to enable the processor 310 to execute one or more applications, such as server applications, network communication processes, and any other type of application or software known to be available on computer systems. Alternatively, the instructions, application programs, etc. can be stored in an external storage or available from a memory over a network. The one or more storage devices can be a volatile or non-volatile, magnetic, semiconductor, tape, optical, removable, non-removable, or other type of storage device or tangible computer-readable medium.

The Monitoring System 320 can include a memory 330 that can include instructions that, when executed by the processor 310, perform one or more processes consistent with the functionalities disclosed herein. Methods, systems, and articles of manufacture consistent with disclosed embodiments are not limited to separate programs or computers configured to perform dedicated tasks. For example, the Monitoring System 320 can include the memory 330 that can include one or more programs 350 to perform one or more functions of the disclosed embodiments. For example, in some embodiments, the Monitoring System 320 can additionally manage dialogue and/or other interactions with the customer via a program 350.

The processor 310 can execute one or more programs 350 located remotely from the Monitoring System 320. For example, the Monitoring System 320 can access one or more remote programs that, when executed, perform functions related to disclosed embodiments.

The memory 330 can include one or more memory devices that store data and instructions used to perform one or more features of the disclosed embodiments. The memory 330 can also include any combination of one or more databases controlled by memory controller devices (e.g., server(s), etc.) or software, such as document management systems, Microsoft™ SQL databases, SharePoint™ databases, Oracle™ databases, Sybase™ databases, or other relational or non-relational databases. The memory 330 can include software components that, when executed by the processor 310, perform one or more processes consistent with the disclosed embodiments. In some embodiments, the memory 330 can include a Monitoring System database 360 for storing related data to enable the Monitoring System 320 to perform one or more of the processes and functionalities associated with the disclosed embodiments.

The Monitoring System database 360 can include stored data relating to status data (e.g., average session duration data, location data, idle time between sessions, and/or average idle time between sessions) and historical status data. According to some embodiments, the functions provided by the Monitoring System database 360 can also be provided by a database that is external to the Monitoring System 320, such as the database 416 as shown in FIG. 14.

The Monitoring System 320 can also be communicatively connected to one or more memory devices (e.g., databases) locally or through a network. The remote memory devices can be configured to store information and can be accessed and/or managed by the Monitoring System 320. By way of example, the remote memory devices can be document management systems, Microsoft™ SQL database, SharePoint™ databases, Oracle™ databases, Sybase™ databases, or other relational or non-relational databases. Systems and methods consistent with disclosed embodiments, however, are not limited to separate databases or even to the use of a database.

The Monitoring System 320 can also include one or more I/O devices 370 that can comprise one or more interfaces for receiving signals or input from devices and providing signals or output to one or more devices that allow data to be received and/or transmitted by the Monitoring System 320. For example, the Monitoring System 320 can include interface components, which can provide interfaces to one or more input devices, such as one or more keyboards, mouse devices, touch screens, track pads, trackballs, scroll wheels, digital cameras, microphones, sensors, and the like, that enable the Monitoring System 320 to receive data from a user (such as, for example, via the user device 402). Additionally, the I/O devices 370 could include a servo motor, imaging device such as a camera, a controller for the motor and/or camera, a ventilator, a perfusion pump and other similar devices, thereby enhancing the system's capability to interact with various medical and monitoring equipment.

In examples of the disclosed technology, the Monitoring System 320 can include any number of hardware and/or software applications that are executed to facilitate any of the operations. The one or more I/O interfaces can be utilized to receive or collect data and/or user instructions from a wide variety of input devices. Received data can be processed by one or more computer processors as desired in various implementations of the disclosed technology and/or stored in one or more memory devices.

The Monitoring System 320 can contain programs that train, implement, store, receive, retrieve, and/or transmit one or more machine learning models. Machine learning models can include a neural network model, a generative adversarial model (GAN), a recurrent neural network (RNN) model, a deep learning model (e.g., a long short-term memory (LSTM) model), a random forest model, a convolutional neural network (CNN) model, a support vector machine (SVM) model, logistic regression, XGBoost, and/or another machine learning model. Models can include an ensemble model (e.g., a model comprised of a plurality of models). In some embodiments, training of a model can terminate when a training criterion is satisfied. Training criterion can include a number of epochs, a training time, a performance metric (e.g., an estimate of accuracy in reproducing test data), or the like. The Monitoring System 320 can be configured to adjust model parameters during training. Model parameters can include weights, coefficients, offsets, or the like. Training can be supervised or unsupervised.

The Monitoring System 320 can be configured to train machine learning models by optimizing model parameters and/or hyperparameters (hyperparameter tuning) using an optimization technique, consistent with disclosed embodiments. Hyperparameters can include training hyperparameters, which can affect how training of the model occurs, or architectural hyperparameters, which can affect the structure of the model. An optimization technique can include a grid search, a random search, a gaussian process, a Bayesian process, a Covariance Matrix Adaptation Evolution Strategy (CMA-ES), a derivative-based search, a stochastic hill-climb, a neighborhood search, an adaptive random search, or the like. The Monitoring System 320 can be configured to optimize statistical models using known optimization techniques.

While the Monitoring System 320 has been described as one form for implementing the techniques described herein, other, functionally equivalent, techniques can be employed. For example, some or all of the functionality implemented via executable instructions can also be implemented using firmware and/or hardware devices such as application specific integrated circuits (ASICs), programmable logic arrays, state machines, etc. Furthermore, other implementations of Monitoring System 320 can include a greater or lesser number of components than those illustrated.

FIG. 14 is a block diagram of an example system 400 that can be used to view and interact with an AI-Platform, according to an example implementation of the disclosed technology. The components and arrangements shown in FIG. 14 are not intended to limit the disclosed embodiments as the components used to implement the disclosed processes and features can vary. As shown, AI-Platform can interact with a user device 402 via a network 406. In certain example implementations, the AI-Platform 408 can include a local network 412, a Monitoring System 320, a web server 410, and a database 416.

In some embodiments, a user can operate the user device 402. The user device 402 can include one or more of a mobile device, smart phone, general purpose computer, tablet computer, laptop computer, telephone, public switched telephone network (PSTN) landline, smart wearable device, voice command device, other mobile computing device, or any other device capable of communicating with the network 406 and ultimately communicating with one or more components of the AI-Platform 408. According to some embodiments, the user device 402 can include an environmental sensor for obtaining audio or visual data, such as a microphone and/or digital camera, an input/output device such as a transceiver for sending and receiving data, a display for displaying digital images, one or more processors, and a memory in communication with the one or more processors.

The network 406 can be of any suitable type, including individual connections via the internet such as cellular or WiFi networks. In some embodiments, the network 406 can connect terminals, services, and mobile devices using direct connections such as radio-frequency identification (RFID), near-field communication (NFC), Bluetooth™, low-energy Bluetooth™ (BLE), WiFi™, ZigBee™, ambient backscatter communications (ABC) protocols, USB, WAN, or LAN. Because the information transmitted can be personal or confidential, security concerns can dictate one or more of these types of connections be encrypted or otherwise secured. In some embodiments, however, the information being transmitted can be less personal, and therefore the network connections can be selected for convenience over security.

The network 406 can include any type of computer networking arrangement used to exchange data. For example, the network 406 can be the Internet, a private data network, virtual private network (VPN) using a public network, and/or other suitable connection(s) that enable(s) components in the system 400 environment to send and receive information between the components of the system 400. The network 406 can also include a PSTN and/or a wireless network.

The AI-Platform 408 can be associated with and optionally controlled by one or more entities such as a business, corporation, individual, partnership, or any other entity that provides one or more of goods, services, and consultations to individuals such as customers. In some embodiments, the AI-Platform 408 can be controlled by a third party on behalf of another business, corporation, individual, partnership. The AI-Platform 408 can include one or more servers and computer systems for performing one or more functions associated with products and/or services that the organization provides.

Web server 410 can include a computer system configured to generate and provide one or more websites accessible to customers, as well as any other individuals involved in access system 408's normal operations. Web server 410 can include a computer system configured to receive communications from user device 402 via for example, a mobile application, a chat program, an instant messaging program, a voice-to-text program, an SMS message, email, or any other type or format of written or electronic communication. Web server 410 can have one or more processors 422 and one or more web server databases 424, which can be any suitable repository of website data. Information stored in web server 410 can be accessed (e.g., retrieved, updated, and added to) via local network 412 and/or network 406 by one or more devices or systems of system 400. In some embodiments, web server 410 can host websites or applications that can be accessed by the user device 402. For example, web server 410 can host a financial service provider website that a user device can access by providing an attempted login that are authenticated by the Monitoring System 320. According to some embodiments, web server 410 can include software tools, similar to those described with respect to user device 402 above, that can allow web server 410 to obtain network identification data from user device 402. The web server can also be hosted by an online provider of website hosting, networking, cloud, or backup services, such as Microsoft Azure™ or Amazon Web Services™.

The local network 412 can include any type of computer networking arrangement used to exchange data in a localized area, such as WiFi, Bluetooth™, Ethernet, and other suitable network connections that enable components of the AI-Platform 408 to interact with one another and to connect to the network 406 for interacting with components in the system 400 environment. In some embodiments, the local network 412 can include an interface for communicating with or linking to the network 406. In other embodiments, certain components of the AI-Platform 408 can communicate via the network 406, without a separate local network 406.

The AI-Platform 408 can be hosted in a cloud computing environment (not shown). The cloud computing environment can provide software, data access, data storage, and computation. Furthermore, the cloud computing environment can include resources such as applications (apps), VMs, virtualized storage (VS), or hypervisors (HYP). User device 402 can be able to access AI-Platform 408 using the cloud computing environment. User device 402 can be able to access AI-Platform 408 using specialized software. The cloud computing environment can eliminate the need to install specialized software on user device 402.

In accordance with certain example implementations of the disclosed technology, the AI-Platform 408 can include one or more computer systems configured to compile data from a plurality of sources the Monitoring System 320, web server 410, and/or the database 416. The Monitoring System 320 can correlate compiled data, analyze the compiled data, arrange the compiled data, generate derived data based on the compiled data, and store the compiled and derived data in a database such as the database 416. According to some embodiments, the database 416 can be a database associated with an organization and/or a related entity that stores a variety of information relating to customers, transactions, ATM, and business operations. The database 416 can also serve as a back-up storage device and can contain data and information that is also stored on, for example, database 360, as discussed with reference to FIG. 13.

As discussed herein, the system 400 can include a perfusion pump 448, which could encompass various types of pumps designed to circulate fluids through tissues or organs, such as roller pumps, centrifugal pumps, or diaphragm pumps. Further, the system 400 can include a ventilator 450, which may include mechanical ventilators, positive pressure ventilators, or negative pressure ventilators used to assist or replace spontaneous breathing. The surgical instrument 452 can refer to a wide range of tools used in surgical procedures, including but not limited to scalpels, forceps, retractors, and electrosurgical devices. The motor 454 can include various types of motors such as servomotors, stepper motors, or brushless DC motors, which are used to drive mechanical components within the system. Additionally, the visual detection subsystem 456 can encompass various imaging and detection technologies, such as cameras, optical sensors, or infrared sensors, which are used to capture and analyze visual information for monitoring or diagnostic purposes.

In some examples, disclosed systems or methods can involve one or more of the following clauses:
Clause 1: A robotic, machine learning system (320, 408) monitoring a live biological material (50), comprising: a visual detection subsystem (456); one or more processors (310); and memory (330) in communication with the one or more processors (310) and storing instructions that, when executed by the one or more processors (310), are configured to cause the system (320, 408) to: receive, at the one or more processors (310) and via the visual detection subsystem (456), an image associated with the live biological material (50); analyze, via the one or more processors (310), the image using a machine learning model to identify a target site (52a, 52b) within the live biological material (50); quantify, via the one or more processors (310), characteristics of the target site (52a, 52b), the quantified characteristics including at least one of cellular density, tissue morphology, or pathological markers; generate, via the one or more processors (310), a real-time visual representation (54) of the quantified characteristics overlaid on the image; continuously monitor, via the one or more processors (310), changes at the target site (52a, 52b) by periodically receiving updated images and reanalyzing the updated images using the machine learning model; provide feedback, via the one or more processors (310), to a user device (402) regarding a status or progression of the live biological material (50) based on the monitored changes; and store data and feedback associated with the quantified characteristics in a database (360, 416) for future reference and further analysis.
Clause 2: The system (320, 408) of Clause 1, wherein the memory (330) stores instructions that, when executed by the one or more processors (310), are configured to cause the system (320, 408) to: receive intraoperative input regarding the at least one of the cellular density, cell viability, tissue morphology, pathological markers, or organ function; and retrain the machine learning model based on the intraoperative input.
Clause 3: The system (320, 408) of Clause 1, wherein the target site (52a, 52b) within the live biological material (50) comprises at least pooled blood or tissue damage.
Clause 4: The system (320, 408) of Clause 1, wherein the target site (52a, 52b) is identified based on light intensity or fluorescence detected in the image.
Clause 5: The system (320, 408) of Clause 4, wherein the target site (52a, 52b) is at least a portion of an in vivo organ, and the system (320, 408) is configured to identify, at the target site (52a, 52b), a dosage of a therapeutic agent received at the target site (52a, 52b) using the light intensity or the fluorescence detected in the image.
Clause 6: The system (320, 408) of Clause 5, wherein the memory (330) stores instructions that, when executed by the one or more processors (310), are configured to train the machine learning model to identify target dosages for a plurality of therapeutics received at a plurality of target organs to identify trends for injection parameters associated with the plurality of therapeutics and the plurality of target organs.
Clause 7: The system (320, 408) of Clause 6, wherein the injection parameters associated with the plurality of therapeutics and plurality of target organs include at least one of dosage, target organ, or injection site.
Clause 8: The system (320, 408) of Clause 1, wherein the memory (330) stores instructions that, when executed by the one or more processors (310), are configured to cause the system (320, 408) to: receive information regarding (i) ventilator or perfusion pump usage data, and (ii) monitoring information related to movement of the live biological material (50); and update the system (320, 408) to account for the movement of the live biological material (50) to predict a location of the live biological material (50) at a particular time based on the ventilator or perfusion pump usage data and the monitoring information.
Clause 9: The system (320, 408) of Clause 1, wherein the memory (330) stores instructions that, when executed by the one or more processors (310), are configured to train the machine learning model on medical imaging, wherein the medical imaging comprises at least one of computed tomography scans (CT scans), magnetic resonance imaging (MRIs), X-rays, ultrasounds, or nuclear medicine.
Clause 10: The system (320, 408) of Clause 9, wherein the memory (330) stores instructions that, when executed by the one or more processors (310), are configured to identify passages through patient anatomy, the passages including at least one of vessels, bronchi, bronchioles, tracheas, or alveoli.
Clause 11: The system (320, 408) of Clause 1, wherein the memory (330) stores instructions that, when executed by the one or more processors (310), are configured to create a registry of information to predict anatomical features of a patient.
Clause 12: The system (320, 408) of Clause 11, wherein the memory (330) stores instructions that, when executed by the one or more processors (310), are configured to: provide operational guidance, based on the registry, during an operation; and output the operational guidance for display on a user device (402).
Clause 13: The system (320, 408) of Clause 1, wherein the memory (330) stores instructions that, when executed by the one or more processors (310), are configured to: train the machine learning model to analyze and store a patient's anatomical data; receive an indication that a surgical instrument (452) has been removed from the target site (52a, 52b) in the patient; and provide operational guidance to a user, when the user re-inserts the surgical instrument (452), to assist in placing the surgical instrument (452) back at the target site (52a, 52b).
Clause 14: The system (320, 408) of Clause 1, wherein the memory (330) stores instructions that, when executed by the one or more processors (310), are configured to output a control signal to a motor to automatically locate and position a surgical instrument (452) at the target site (52a, 52b) within the live biological material (50).
Clause 15: A method for using data for a live biological material (50) to automatically assist a surgical instrument (452) to locate and position itself at a target site (52a, 52b), the method comprising: receiving, at one or more processors (310), an image associated with the live biological material (50) via a visual detection subsystem (456); analyzing, via the one or more processors (310), the image using a machine learning model to identify the target site (52a, 52b) within the live biological material (50); quantifying, via the one or more processors (310), characteristics of the target site (52a, 52b), the quantified characteristics including at least one of cellular density, tissue morphology, or pathological markers; continuously monitoring, via the one or more processors (310), changes at the target site (52a, 52b) by periodically receiving updated images and reanalyzing the updated images using the machine learning model; outputting, via the one or more processors (310), control signals to a motorized system (320, 408) based on the image to automatically instruct the motorized system (320, 408) to locate and position a surgical instrument (452) at the target site (52a, 52b) within the live biological material (50); and adjusting, via the one or more processors (310), the position of the surgical instrument (452) in real-time based on the continuous monitoring and updated analysis of the target site (52a, 52b).
Clause 16: The method of Clause 15 further comprising: receiving, at the one or more processors (310), information regarding (i) ventilator or perfusion pump usage data, and (ii) monitoring information related to movement of the live biological material (50); and updating, via the one or more processors (310), the system (320, 408) to account for the movement of the live biological material (50) to predict a location of the live biological material (50) at a particular time based on the ventilator or perfusion pump usage data and the monitoring information.
Clause 17: The method of Clause 15 further comprising: training the machine learning model to analyze and store a person's anatomical data; receiving, via the one or more processors (310), an indication that a surgical instrument (452) has been removed from the target site (52a, 52b) in the person; and providing operational guidance, via the one or more processors (310), to a user when the user re-inserts the surgical instrument (452) to assist in placing the surgical instrument (452) back at the target site (52a, 52b).
Clause 18: A method for monitoring a subject patient's anatomy during a surgery and assisting in re-delivery of a surgical instrument (452) to a target site (52a, 52b), the method comprising: monitoring, via one or more processors (310), the subject patient's anatomy during the surgery using a visual detection subsystem (456); receiving, at the one or more processors (310), an indication that a surgical instrument (452) has been removed from the target site (52a, 52b) within the patient; analyzing, via the one or more processors (310), the monitored anatomy to determine a location of the target site (52a, 52b) from which the surgical instrument (452) was removed; providing operational guidance, via the one or more processors (310), to a user device (402) to assist a user in delivering the surgical instrument (452) back into the same target site (52a, 52b) within the patient; and adjusting, via the one or more processors (310), the operational guidance in real-time based on continuous monitoring and updated analysis of the subject patient's anatomy.
Clause 19: The method of Clause 18 further comprising: creating, via the one or more processors (310), a registry of information to predict anatomical features of one or more patients; and updating the registry with information related to the updated analysis of the subject patient's anatomy.
Clause 20: The method of Clause 18 further comprising: receiving, at the one or more processors (310), monitoring information related to movement of the subject patient's anatomy; and updating, via the one or more processors (310), a machine learning model to account for the movement of the subject patient's anatomy to predict a location of one or more tissues or organs at a particular time based on the monitoring information.

The features and other aspects and principles of the disclosed embodiments can be implemented in various environments. Such environments and related applications can be specifically constructed for performing the various processes and operations of the disclosed embodiments or they can include a general-purpose computer or computing platform selectively activated or reconfigured by program code to provide the necessary functionality. Further, the processes disclosed herein can be implemented by a suitable combination of hardware, software, and/or firmware. For example, the disclosed embodiments can implement general purpose machines configured to execute software programs that perform processes consistent with the disclosed embodiments. Alternatively, the disclosed embodiments can implement a specialized apparatus or system configured to execute software programs that perform processes consistent with the disclosed embodiments. Furthermore, although some disclosed embodiments can be implemented by general purpose machines as computer processing instructions, all or a portion of the functionality of the disclosed embodiments can be implemented instead in dedicated electronics hardware.

The disclosed embodiments also relate to tangible and non-transitory computer readable media that include program instructions or program code that, when executed by one or more processors, perform one or more computer-implemented operations. The program instructions or program code can include specially designed and constructed instructions or code, and/or instructions and code well-known and available to those having ordinary skill in the computer software arts. For example, the disclosed embodiments can execute high level and/or low-level software instructions, such as machine code (e.g., such as that produced by a compiler) and/or high-level code that can be executed by a processor using an interpreter.

The technology disclosed herein typically involves a high-level design effort to construct a computational system that can appropriately process unpredictable data. Mathematical algorithms can be used as building blocks for a framework, however certain implementations of the system can autonomously learn their own operation parameters, achieving better results, higher accuracy, fewer errors, fewer crashes, and greater speed.

As used in this application, the terms “component,” “module,” “system,” “server,” “processor,” “memory,” and the like are intended to include one or more computer-related units, such as but not limited to hardware, firmware, a combination of hardware and software, software, or software in execution. For example, a component can be, but is not limited to being, a process running on a processor, an object, an executable, a thread of execution, a program, and/or a computer. By way of illustration, both an application running on a computing device and the computing device can be a component. One or more components can reside within a process and/or thread of execution and a component can be localized on one computer and/or distributed between two or more computers. In addition, these components can execute from various computer readable media having various data structures stored thereon. The components can communicate by way of local and/or remote processes such as in accordance with a signal having one or more data packets, such as data from one component interacting with another component in a local system, distributed system, and/or across a network such as the Internet with other systems by way of the signal.

Certain embodiments and implementations of the disclosed technology are described above with reference to block and flow diagrams of systems and methods and/or computer program products according to example embodiments or implementations of the disclosed technology. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, can be repeated, or may not necessarily need to be performed at all, according to some embodiments or implementations of the disclosed technology.

These computer-executable program instructions can be loaded onto a general-purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions can also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks.

As an example, embodiments or implementations of the disclosed technology can provide for a computer program product, including a computer-usable medium having a computer-readable program code or program instructions embodied therein, said computer-readable program code adapted to be executed to implement one or more functions specified in the flow diagram block or blocks. Likewise, the computer program instructions can be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special-purpose hardware and computer instructions.

Certain implementations of the disclosed technology described above with reference to user devices can include mobile computing devices. Those skilled in the art recognize that there are several categories of mobile devices, generally known as portable computing devices that can run on batteries but are not usually classified as laptops. For example, mobile devices can include, but are not limited to portable computers, tablet PCs, internet tablets, PDAs, ultra-mobile PCs (UMPCs), wearable devices, and smart phones. Additionally, implementations of the disclosed technology can be utilized with internet of things (IoT) devices, smart televisions and media devices, appliances, automobiles, toys, and voice command devices, along with peripherals that interface with these devices.

In this description, numerous specific details have been set forth. It is to be understood, however, that implementations of the disclosed technology can be practiced without these specific details. In other instances, well-known methods, structures, and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described can include a particular feature, structure, or characteristic, but not every implementation necessarily includes the particular feature, structure, or characteristic. Further, repeated use of the phrase “in one implementation” does not necessarily refer to the same implementation, although it can.

Throughout the specification and the claims, the following terms take at least the meanings explicitly associated herein, unless the context clearly dictates otherwise. The term “connected” means that one function, feature, structure, or characteristic is directly joined to or in communication with another function, feature, structure, or characteristic. The term “coupled” means that one function, feature, structure, or characteristic is directly or indirectly joined to or in communication with another function, feature, structure, or characteristic. The term “or” is intended to mean an inclusive “or.” Further, the terms “a,” “an,” and “the” are intended to mean one or more unless specified otherwise or clear from the context to be directed to a singular form. By “comprising” or “containing” or “including” is meant that at least the named element, or method step is present in article or method, but does not exclude the presence of other elements or method steps, even if the other such elements or method steps have the same function as what is named.

It is to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Although embodiments are described herein with respect to systems or methods, it is contemplated that embodiments with identical or substantially similar features can alternatively be implemented as systems, methods and/or non-transitory computer-readable media.

As used herein, unless otherwise specified, the use of the ordinal adjectives “first,” “second,” “third,” etc., to describe a common object, merely indicates that different instances of like objects are being referred to, and is not intended to imply that the objects so described must be in a given sequence, either temporally, spatially, in ranking, or in any other manner.

While certain embodiments of this disclosure have been described in connection with what is presently considered to be the most practical and various embodiments, it is to be understood that this disclosure is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the technology and also to enable any person skilled in the art to practice certain embodiments of this technology, including making and using any apparatuses or systems and performing any incorporated methods. The patentable scope of certain embodiments of the technology is defined in the claims, and can include other examples that occur to those skilled in the art. Such other examples are intended to be within the scope of the claims if they have structural elements that do not differ from the literal language of the claims, or if they include equivalent structural elements with insubstantial differences from the literal language of the claims.

### ILLUSTRATIVE EXAMPLES

The following examples illustrate certain embodiments of the disclosed technology and are not intended to be limiting.

### Example 1: Ex Vivo Lung Monitoring and Cell Tracking

In one illustrative implementation, ex vivo lungs are maintained on a ventilator and perfusion system for assessment and rehabilitation prior to transplantation. The visual detection subsystem, comprising one or more cameras positioned around the lungs, continuously captures images of the lung tissue. Fluorescently labeled therapeutic cells, such as mesenchymal stem cells, are injected into the lungs via bronchoscopic delivery. The AI-driven system detects the fluorescently labeled cells based on light intensity, tracks their distribution across the lung tissue, and counts the cells in real-time using the machine learning model. The system correlates the detected cell count with the intended therapeutic dosage, providing feedback to the user device indicating whether the target dosage has been achieved. The system may also monitor changes in cell count over time to track cell retention, migration, or proliferation at the target site. All data, including images, cell counts, and dosage information, is stored in the database for future reference and analysis, enabling retrospective evaluation of therapeutic outcomes.

### Example 2: AI-Driven Bronchoscopic Navigation

In another illustrative implementation, a robotic bronchoscope is used to navigate to a target region within a patient's lungs for biopsy or targeted drug delivery. Prior to the procedure, the system is trained on pre-operative imaging data, such as CT scans, to map the patient's airway anatomy and plan a navigation path to the target lung region. During the procedure, the robotic bronchoscope is inserted into the patient's airway, and the visual detection subsystem captures real-time images of the airway passages. At each bifurcation point, the system acquires images of the available branch airways, performs shape extraction to isolate the geometric contours of each airway, and uses the machine learning model to identify each branch and classify it as target or non-target. The system determines the spatial coordinates of the target airway and outputs control signals to the motor to automatically guide the bronchoscope toward the target branch. This process repeats at each subsequent bifurcation until the instrument reaches the target lung region. The system stores the navigation path as a sequence of bifurcation decisions. If the bronchoscope is removed and reinserted, the system provides operational guidance to assist in re-navigating to the same target site based on the stored anatomical data and navigation path.

What is claimed is:

1. A robotic, machine learning system monitoring a live biological material, comprising:
a visual detection subsystem;
one or more processors; and
memory in communication with the one or more processors and storing instructions that, when executed by the one or more processors, are configured to cause the system to:
receive, at the one or more processors and via the visual detection subsystem, an image associated with the live biological material;
analyze, via the one or more processors, the image using a machine learning model to identify a target site within the live biological material;
quantify, via the one or more processors, characteristics of the target site, the quantified characteristics including at least one of cellular density, tissue morphology, or pathological markers;
generate, via the one or more processors, a real-time visual representation of the quantified characteristics overlaid on the image;
continuously monitor, via the one or more processors, changes at the target site by periodically receiving updated images and reanalyzing the updated images using the machine learning model;
provide feedback, via the one or more processors, to a user device regarding a status or progression of the live biological material based on the monitored changes; and
store data and feedback associated with the quantified characteristics in a database for future reference and further analysis.

2. The system of claim 1, wherein the memory stores instructions that, when executed by the one or more processors, are configured to cause the system to:
receive intraoperative input regarding the at least one of the cellular density, cell viability, tissue morphology, pathological markers, or organ function; and
retrain the machine learning model based on the intraoperative input.

3. The system of claim 1, wherein the target site within the live biological material comprises at least pooled blood or tissue damage.

4. The system of claim 1, wherein the target site is identified based on light intensity or fluorescence detected in the image.

5. The system of claim 4, wherein the target site is at least a portion of an in vivo organ, and the system is configured to identify, at the target site, a dosage of a therapeutic agent received at the target site using the light intensity or the fluorescence detected in the image.

6. The system of claim 5, wherein the memory stores instructions that, when executed by the one or more processors, are configured to train the machine learning model to identify target dosages for a plurality of therapeutics received at a plurality of target organs to identify trends for injection parameters associated with the plurality of therapeutics and the plurality of target organs.

7. The system of claim 6, wherein the injection parameters associated with the plurality of therapeutics and plurality of target organs include at least one of dosage, target organ, or injection site.

8. The system of claim 1, wherein the memory stores instructions that, when executed by the one or more processors, are configured to cause the system to:
receive information regarding (i) ventilator or perfusion pump usage data, and (ii) monitoring information related to movement of the live biological material; and
update the system to account for the movement of the live biological material to predict a location of the live biological material at a particular time based on the ventilator or perfusion pump usage data and the monitoring information.

9. The system of claim 1, wherein the memory stores instructions that, when executed by the one or more processors, are configured to train the machine learning model on medical imaging, wherein the medical imaging comprises at least one of computed tomography scans (CT scans), magnetic resonance imaging (MRIs), X-rays, ultrasounds, or nuclear medicine.

10. The system of claim 9, wherein the memory stores instructions that, when executed by the one or more processors, are configured to identify passages through patient anatomy, the passages including at least one of vessels, bronchi, bronchioles, tracheas, or alveoli.

11. The system of claim 1, wherein the memory stores instructions that, when executed by the one or more processors, are configured to create a registry of information to predict anatomical features of a patient.

12. The system of claim 11, wherein the memory stores instructions that, when executed by the one or more processors, are configured to:
provide operational guidance, based on the registry, during an operation; and
output the operational guidance for display on a user device.

13. The system of claim 1, wherein the memory stores instructions that, when executed by the one or more processors, are configured to:
train the machine learning model to analyze and store a patient's anatomical data;
receive an indication that a surgical instrument has been removed from the target site in the patient; and
provide operational guidance to a user, when the user re-inserts the surgical instrument, to assist in placing the surgical instrument back at the target site.

14. The system of claim 1, wherein the memory stores instructions that, when executed by the one or more processors, are configured to output a control signal to a motor to automatically locate and position a surgical instrument at the target site within the live biological material.

15. A method for using data for a live biological material to automatically guide a surgical instrument to locate and position itself at a target site, the method comprising:
receiving, at one or more processors, an image associated with the live biological material via a visual detection subsystem;
analyzing, via the one or more processors, the image using a machine learning model to identify the target site within the live biological material;
quantifying, via the one or more processors, characteristics of the target site, the quantified characteristics including at least one of cellular density, tissue morphology, or pathological markers;
continuously monitoring, via the one or more processors, changes at the target site by periodically receiving updated images and reanalyzing the updated images using the machine learning model;
outputting, via the one or more processors, control signals to a motorized system based on the image to automatically instruct the motorized system to locate and position a surgical instrument at the target site within the live biological material; and
adjusting, via the one or more processors, the position of the surgical instrument in real-time based on the continuous monitoring and updated analysis of the target site.

16. The method of claim 15 further comprising:
receiving, at the one or more processors, information regarding (i) ventilator or perfusion pump usage data, and (ii) monitoring information related to movement of the live biological material; and
updating, via the one or more processors, the system to account for the movement of the live biological material to predict a location of the live biological material at a particular time based on the ventilator or perfusion pump usage data and the monitoring information.

17. The method of claim 15 further comprising:
training the machine learning model to analyze and store a person's anatomical data;
receiving, via the one or more processors, an indication that a surgical instrument has been removed from the target site in the person; and
providing operational guidance, via the one or more processors, to a user when the user re-inserts the surgical instrument to assist in placing the surgical instrument back at the target site.

18. A method for monitoring a subject patient's anatomy during a surgery and assisting in re-delivery of a surgical instrument to a target site, the method comprising:
monitoring, via one or more processors, the subject patient's anatomy during the surgery using a visual detection subsystem;
receiving, at the one or more processors, an indication that a surgical instrument has been removed from the target site within the patient;
analyzing, via the one or more processors, the monitored anatomy to determine a location of the target site from which the surgical instrument was removed;
providing operational guidance, via the one or more processors, to a user device to assist a user in delivering the surgical instrument back into the same target site within the patient; and
adjusting, via the one or more processors, the operational guidance in real-time based on continuous monitoring and updated analysis of the subject patient's anatomy.

19. The method of claim 18 further comprising:
creating, via the one or more processors, a registry of information to predict anatomical features of one or more patients; and
updating the registry with information related to the updated analysis of the subject patient's anatomy.

20. The method of claim 18 further comprising:
receiving, at the one or more processors, monitoring information related to movement of the subject patient's anatomy; and
updating, via the one or more processors, a machine learning model to account for the movement of the subject patient's anatomy to predict a location of one or more tissues or organs at a particular time based on the monitoring information.
